(19) United States
(12) Patent Application Publication
(10) Pub. No.: US 2026/0091055 A1
(43) Pub. Date: Apr. 2, 2026

(54) Methods of Managing Traumatic Brain Injury

(71) Applicants: Emory University, Atlanta, GA (US); Yale University, New Haven, CT (US)

(72) Inventors: David Gimbel, Atlanta, GA (US); Stephen M. Strittmatter, Durham, CT (US)

(21) Appl. No.: 19/111,224

(22) PCT Filed: Sep. 12, 2023
(86) PCT No.: PCT/US2023/032519

§ 371 Date: Mar. 12, 2025

Related U.S. Application Data
(60) Provisional application No. 63/405,622, filed on Sep. 12, 2022.

(51) Int. Cl. A61K 31/795; A61K 31/192; A61K 31/366; A61K 31/405; A61K 31/573; A61K 31/635; A61P 25/00

(52) U.S. Cl. CPC A61K 31/795; A61K 31/192; A61K 31/366; A61K 31/405; A61K 31/573; A61K 31/635; A61P 25/00

(57) ABSTRACT

This disclosure relates to methods of treating or preventing traumatic brain injury (TBI) or related conditions comprising administering an effective amount of poly [4-styrenesulfonic acid-co-maleic acid] (PSCMA) or alternative anionic polymer or a pharmaceutically acceptable salt thereof to a subject in need thereof. In certain embodiments, this disclosure relates to methods of treating chronic traumatic encephalopathy (CTE) comprising administering an effective amount of poly [4-styrenesulfonic acid-co-maleic acid] (PSCMA) or alternative anionic polymer, or a pharmaceutically acceptable salt thereof to subject in need thereof.

### CROSS-REFERENCE TO RELATED APPLICATIONS

This application claims the benefit of U.S. Provisional Application No. 63/405,622 filed Sep. 12, 2022. The entirety of this application is hereby incorporated by reference for all purposes.

### BACKGROUND

Traumatic brain injury can result in symptoms such as headaches and temporary confusion, or in more severe cases coma or even death. Concussions are non-penetrating or blunt injuries that result in temporary loss of normal brain function. Concussions are sometimes a result of a blow to the head or a jolt causing whiplash, which may knock the brain against the inside of the skull. Symptoms often last for only a short period. Chronic traumatic encephalopathy (CTE) is a progressive brain condition caused by repeated blows to the head and repeated episodes of concussion which commonly results in lasting damage to the brain. Standard treatments with inflammatory agents are not universally effective in preventing long-term complications. Thus, there is a need to identify improved therapies.

Gunther et al. report the rescue of transgenic Alzheimer's pathophysiology by polymeric cellular prion protein antagonists. Cell Rep, 2019, 26(1):145-158. See also US Patent Publication 2021/0268016.

Sagare et al. report prion protein antagonists rescue Alzheimer's amyloid-beta-related cognitive deficits. Trends Mol Med, 2019, 25(2):74-76.

Rubenstein et al. report tau phosphorylation induced by severe closed head traumatic brain injury is linked to the cellular prion protein. Acta Neuropathologica Communications, 2017, 5:30.

Vink reports methods of preventing or treating chronic traumatic encephalopathy. See US Patent Publication 2022/0218663.

References cited herein are not an admission of prior art.

### SUMMARY

This disclosure relates to methods of treating or preventing traumatic brain injury or related conditions comprising administering an effective amount of poly [4-styrenesulfonic acid-co-maleic acid] (PSCMA) or alternative anionic polymer or a pharmaceutically acceptable salt thereof to a subject in need thereof. In certain embodiments, this disclosure relates to methods of treating chronic traumatic encephalopathy comprising administering an effective amount of poly [4-styrenesulfonic acid-co-maleic acid] (PSCMA) or alternative anionic polymer, or a pharmaceutically acceptable salt thereof to subject in need thereof.

In certain embodiments, the subject experienced a concussion and was unconscious for more than one second, three seconds, or for more than 10 seconds. In certain embodiments, the subject does not have a neurodegenerative disease such as Alzheimer's disease. In certain embodiments, PSCMA is administered in combination with a second therapeutic agent such as an anti-inflammatory agent.

### BRIEF DESCRIPTION OF THE SEVERAL VIEWS OF THE DRAWINGS

FIG. 1 shows data indicating deletion of cellular PrP protects against memory impairment in a mouse CTE model.

FIG. 2A shows in vivo data in mice using poly(4-styrenesulfonic acid-co-maleic acid) sodium salt (PSCMA) indicating pretreatment with PSCMA (P) protects against repetitive head injuries (RHI).

FIG. 2B shows percentage of time in target quadrant using PSCMA (P).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. “Embodiments” refer to an example, and it is contemplated that the embodiments are not necessarily limited to the example.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent. Also, the term “or” is used in its inclusive sense (and not in its exclusive sense) so that when used, for example, to connect a list of elements, the term “or” means one, some, or all of the elements in the list.

As used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

The terms “comprising,” “including,” “having,” and the like are synonymous and are used inclusively, in an open-ended fashion, and do not exclude additional elements, features, acts, operations, and so forth. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

A “Subject” refers to any animal, preferably a human patient, livestock, rodent, monkey, or domestic pet.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

As used herein, the terms “prevention” and “preventing” are used herein to refer to an approach for obtaining beneficial or desired results including, but not limited, to prophylactic benefit. For prophylactic benefit, the pharmaceutical compositions can be administered to a patient at risk of developing a particular disease, or to a patient reporting one or more of the physiological symptoms of a disease, even though a diagnosis of this disease may not have been made.

The term “effective amount” is the quantity which when delivered, improves the prognosis of the subject. The amount to be delivered will depend on the particular characteristics of the condition being treated, the mode of delivery, and the characteristics of the subject, such as general health, other diseases, age, sex, genotype, body weight and tolerance to drugs.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other such that they are contained/circulating in the patient at the same time, e.g., considering half-lives.

As used herein, the term “TBI” or “traumatic brain injury” refers to a condition in which the brain has been subjected to a rapid acceleration/deceleration, blast wave, and/or penetrating injury, which may be accompanied by loss of consciousness and/or retrograde amnesia. The Glasgow Coma Scale (GCS) classifies traumatic brain injuries as mild (14-15), moderate (9-13), or severe (3-8), and all TBIs falling in that range of scores are considered to fall within the scope of this disclosure.

### Methods of Treating or Preventing Traumatic Brain Injury and Related Conditions Using Compositions of Poly [4-styrenesulfonic acid-co-maleic acid] (PSCMA) or Alternative Anionic Polymers

Traumatic brain injury (TBI) can be triggered by a variety of causes. Common causes include falls, being struck by an object, motor vehicle crashes, blast wave exposure (e.g., in military veterans), and contact in sports activities. Traumatic brain injury is further characterized by its severity: mild traumatic brain injury (mTBI), such as a concussion, typically results in symptoms that last for a relatively short period of time, while severe traumatic brain injury generally results in an extended period of negative effects. Common symptoms of TBI include memory lapse, loss of consciousness, headaches, impaired movement, compromised senses, dementia, depression, or other emotional changes. Such brain dysfunction may be apparent immediately after the traumatic event or it may be apparent within hours or days of the traumatic event.

In certain embodiments, TBI may occur as a result of a fall, participation in a high-risk sport such as football, hockey, soccer, rugby, boxing or other contact sport, involvement in a motor vehicle collision either as a passenger or pedestrian (by-stander), involvement in a bicycle collision either as a rider or a pedestrian (by-stander), involvement in combat (e.g., as a solider or by-stander), exposure to, including close proximity to, bomb blasts, physical abuse such as violent head shaking or blows to the head, skull penetration of an object such as a bullet or shrapnel or shattered skull, and the like.

TBI may be diagnosed by the presence of one or more TBI-related symptoms and/or by imaging of the brain, typically following the occurrence of a traumatic event. Such symptoms may first arise within a week to a few weeks or few months of the traumatic event. Similarly, these symptoms may persist for days, weeks, or months following the traumatic event.

TBI-related symptoms include headache or sensation of pressure in the head, temporary loss of consciousness, confusion, amnesia surrounding the traumatic event giving rise to the TBI, dizziness, ringing in the ears, nausea, vomiting, slurred speech, delayed responsiveness (e.g., delayed response to questions), appearing dazed, fatigue, pupil dilation, compromised vision, and difficulty breathing. One or more symptoms may arise immediately after the traumatic event, or they may arise within hours or even days of the traumatic event. Delayed symptoms may include, but are not limited to, concentration and memory deficiencies, irritability and/or other personality changes, sensitivity to light and/or sound, changes in sleep patterns, changes to ability to taste and/or smell, and psychological adjustment issues and depression.

TBI may be mild, moderate, or severe, depending on the number, severity, and duration of symptoms. Mild TBI is typically associated with temporary brain dysfunction. Severe TBI may be associate with bruising, torn tissues, bleeding, and other physical damage to the brain. Certain TBI may be associated with concussions. Concussions typically refer to non-structural, typically non-hemorrhaging, injuries of the brain.

In certain embodiments, methods provided herein are intended to provide therapeutic benefit to subjects that have experienced a TBI. Such therapeutic benefit may impact short term sequelae of the TBI and/or they may impact the long-term sequelae of the TBI. An example of a long-term sequelae of a TBI is believed to be chronic traumatic encephalopathy (CTE). CTE refers to a condition characterized by progressive brain deterioration and caused by one and typically repeated TBIs. An example of CTE is another condition referred to as dementia pugilistica (DP) which tends to be diagnosed in those with a history of boxing. Hallmark symptoms associated with CTE usually manifest themselves several years after the occurrence of the TBIs. These symptoms include deterioration in attention as well as disorientation, dizziness, and headaches. As the condition progresses, memory loss, social instability, erratic behavior, and poor judgement are also apparent. The latter stages of the condition involve progressive dementia, slowing of muscular movements, impeded speech, tremors, vertigo, deafness, and suicidal tendencies. The methods provided herein which are geared towards early intervention following a TBI are expected to benefit TBI subjects both in the short-term as well as in the long-term including for example by reducing their risk of developing CTE, delaying the manifestation of CTE, and/or reducing the severity of CTE if and when it does develop. The short-term readouts, such as the short-term symptoms associated with TBI, in a sense may act as surrogates for the ability to impact the progression of CTE in such subjects.

In certain embodiments, this disclosure relates to methods of treating or preventing TBI, CTE, dementia, memory loss, or related complications comprising administering an effective amount of poly [4-styrenesulfonic acid-co-maleic acid] (PSCMA) or alternative anionic polymer, or a pharmaceutically acceptable salt thereof to a subject in need thereof. In certain embodiments, the subject is a human patient.

In certain embodiments, this disclosure relates to methods of lessening the short-term impact of such TBI, CTE, and/or reducing the risk of developing and/or the severity of long-term aftereffects. In certain embodiments, this disclosure relates to methods of early and optionally repeated use can decrease the severity of acute TBI, CTE, reduce the risk of developing, delay the onset of, and/or reduce the severity of TBI, CTE sequelae. In certain embodiments, the subject may have experienced a concussive TBI (i.e., the subject has experienced a concussion).

In certain embodiments, this disclosure relates to methods of preventing or treating sequelae of TBI which are cognitive and/or behavioral dysfunction, motor neural dysfunction (coordination) and/or vestibular dysfunction (balance), and/or cerebral inflammation and/or hyperglycemia; temporary confusion, blurry vision, difficulty concentrating, cognitive dysfunction (e.g., confusion, loss of attention, memory, recall, executive function, etc.), behavioral dysfunction (e.g., depression, anxiety, moodiness, etc.), motor neural dysfunction (e.g., weakness, loss in body coordination or controlled movement, etc.), vestibular dysfunction (e.g., loss or impairment of balance), seizures, pain (especially headache), consciousness, impaired cognition, impaired cognitive processing speed, impaired language, impaired motor activity, impaired memory, impaired motor skills, impaired sensory skills, cerebral ischemia, edema, intracranial pressure, hearing loss, tinnitus, decreased smell or taste, reduced strength, coma, or death in a subject that has experienced a traumatic brain injury comprising administering an effective amount of poly [4-styrenesulfonic acid-co-maleic acid] (PSCMA) or alternative anionic polymer, or a pharmaceutically acceptable salt thereof to the subject.

In certain embodiments, the subject that has experienced a TBI will typically manifest one or more of the following symptoms including but not limited to headache or sensation of pressure in the head, temporary loss of consciousness, confusion, amnesia surrounding the traumatic event giving rise to the TBI, dizziness, ringing in the ears, nausea, vomiting, slurred speech, delayed responsiveness (e.g., delayed response to questions), appearing dazed, fatigue, pupil dilation, compromised vision, and difficulty breathing. One or more symptoms may arise immediately after the traumatic event, or they may arise within hours or even days of the traumatic event. Delayed symptoms may include, but are not limited to, concentration and memory deficiencies, irritability and/or other personality changes, sensitivity to light and/or sound, changes in sleep patterns, changes to ability to taste and/or smell, and psychological adjustment issues and depression. Typically, at the time of treatment, the subject does not manifest any long-term effects of a TBI such as symptoms associated with chronic traumatic encephalopathy.

In certain embodiments, the subject typically does not have a tauopathy, as may be determined by medical imaging such as PET imaging for tau tangles and/or a collection of symptoms associated with a tauopathy. In certain embodiments, the subject typically also does not have a neurodegenerative disease such as but not limited to Alzheimer's disease. Those of ordinary skill in the medical arts are aware of the symptoms and physiological manifestations of neurodegenerative diseases such as Alzheimer's including brain mass abnormalities, presence and/or accumulation of beta-amyloid plaques, and the like.

In certain embodiments, the concussion itself will be diagnosed by the presence of one or more of the foregoing symptoms. The concussion may be mild, moderate, or severe.

In certain embodiments, the subject experienced head trauma and loss of consciousness for a more than one second, three seconds, one minute, or one hour.

In some instances, the subject has experienced one or more previous TBIs.

In certain embodiments, this disclosure relates to methods of treating chronic traumatic encephalopathy (CTE) comprising administering an effective amount of poly [4-styrenesulfonic acid-co-maleic acid] (PSCMA) or alternative anionic polymer, or a pharmaceutically acceptable salt thereof to a subject in need thereof. In certain embodiments, the subject is a human patient.

In certain embodiments, the poly [4-styrenesulfonic acid-co-maleic acid] (PSCMA) or alternative anionic polymer, or a pharmaceutically acceptable salt thereof is administered daily, e.g., for 2, 3, or 4 days, a week, one month or more.

In certain embodiments, this disclosure relates to methods for treating a concussive injury which involves a patient being exposed to multiple (more than one) concussive events. In such a method, the attendant physician would determine that the subject is concussed and that the subject has had at least one previous concussion. Methods for determining whether or not a subject has been concussed includes for instance a variety of neuropsychological assessment tools. However, the detection of loss of memory, an alteration of mental state (mental cloudiness, headache, dizziness, confusion, disorientation), possible loss of consciousness, or focal neurological deficits may be used for on-field diagnosis of a concussive event.

In certain embodiments, this disclosure relates to methods for treating a subject which has been exposed to multiple concussive events including the step of administering to the subject poly [4-styrenesulfonic acid-co-maleic acid] (PSCMA) or alternative anionic polymer, or a pharmaceutically acceptable salt thereof as a single oral dose in an amount which is able to maintain the blood concentration of the poly [4-styrenesulfonic acid-co-maleic acid] (PSCMA) or other an anionic polymer, or a pharmaceutically acceptable salt thereof in the therapeutic range for at least 1 or 2 days, wherein the administration step is performed before and/or after the second concussive event and again after each additional concussive event.

In certain embodiments, this disclosure relates to methods of prophylactic neuroprotection that prevents or reduces sequelae of a concussion or other traumatic brain injury, comprising administering poly [4-styrenesulfonic acid-co-maleic acid] (PSCMA) or alternative anionic polymer, or a pharmaceutically acceptable salt thereof prior to a high-risk event, e.g., sports event, military operation. In certain embodiments, the method further comprises administering at least one additional dosage unit of the composition upon the occurrence of a concussion or other traumatic brain injury.

In certain embodiments, the poly [4-styrenesulfonic acid-co-maleic acid] (PSCMA) or alternative anionic polymer, or a pharmaceutically acceptable salt thereof is administered within 24 hours before or after a concussive event or a high-risk event. In an embodiment administration is provided within 20 hours such as within, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours and within 1 hour, of the concussive event or high-risk event.

In some embodiments, the step of identifying the subject having a traumatic brain injury comprises conducting a computerized tomography (CT) scan or magnetic resonance imagining (MRI) on the subject. In some embodiments, the subject is identified as having a traumatic brain injury based on a modified neurological severity score (mNSS), a rotarod test, a Glasgow Coma Scale score, or a Glasgow Outcome Scale score. In some embodiments, the mNSS or the Glasgow Coma Scale score is between 3 and 8, between 9 and 12, or between 13 and 15.

TBI may be diagnosed following a computer tomography (CT) scan which detects brain tissue damage or intracranial lesions. Various blood proteins have been linked to the presence of symptoms of TBI, including ubiquitin carboxy-terminal hydrolase L1 (UCH-L1), glial fibrillary acidic protein (GFAP) and calcitonin gene related peptide (CGRP). In certain embodiments, the step of identifying a subject having a traumatic brain injury comprises: determining a level of a biomarker in a sample from the subject; comparing the level of the biomarker to a reference level of the biomarker; and identifying the subject as having the traumatic brain injury if the determined level of the biomarker is increased as compared to the reference level of the biomarker. In some embodiments, the biomarker can be IL-1beta, TNF-alpha, and IFN-gamma, IL-6, IL-10, IL-8, HMGB1, TGFbeta, UCH-L1, NSE, GFAP, S100B, NF proteins (L and H), MBP, Tau, or phospho-Tau. In some embodiments, the therapeutically effective amount of poly [4-styrenesulfonic acid-co-maleic acid] (PSCMA) or alternative anionic polymer is determined so that the poly [4-styrenesulfonic acid-co-maleic acid] (PSCMA) or alternative anionic polymer increases or decreases the level of the biomarker by at least 5%, 10%, 20%, 30%, 40%, or 50%.

In certain embodiments, poly [4-styrenesulfonic acid-co-maleic acid] (PSCMA) or alternative anionic polymer, or a pharmaceutically acceptable salt thereof in the form of a tablet, pill, capsule, drink solution, or other delivery composition. In certain embodiments, administration and delivery of the compositions may be for example oral, parenteral, or intravenous.

In certain embodiments, poly [4-styrenesulfonic acid-co-maleic acid] (PSCMA) or alternative anionic polymer, or a pharmaceutically acceptable salt thereof is administered in combination with a second therapeutic agent such as an anti-inflammatory agent, glucocorticoid, dexamethasone, progesterone, methylprednisolone, a non-steroidal anti-inflammatory drug, indomethacin, ibuprofen, carprofen, celecoxib, a TNFalpha antagonist, etanercept, anakinra, a phosphodiesterase inhibitor, rolipram, minocycline, erythropoietin, a statin, simvastatin, atorvastatin, lovastatin, N-acetylcysteine, a hemostatic drug, an antifibrinolytic, aprotinin, tranexamic acid (TXA), aminocaproic acid or recombinant activated factor VIIa (rFVIIa).

In certain embodiments, anti-inflammatory agent can be selected from the group consisting of aceclofenac, acemetacin, acetyl-salicylic acid, 5-aminoacetyl salicylic acid, alclofenac, amfenac, bendazac, benoxaprofen, bermoprofen, 5-bromo salicylic acid acetate, butibufen, caffeic acid, carprofen, cinmetacin, clidanac, clopirac, sodium diclofenac, diflunisal, 3,4-dihydroxybenzoic acid, etodolac, felbinac, fenbufen, fendosal, fenoprofen, fentiazac, flufenamic acid, flunixin, flunoxaprofen, flurbiprofen, 1-hydroxynaphthoic acid, ibuprofen, indomethacin, indoprofen, isoxepac, ketoprofen, ketorolac, loxoprofen, meclofenamic acid, mefenamic acid, 3,4-methylenedioxycinnamic acid, montelukast, mycophenolic acid, naproxen, niflumic acid, olsalazine, oxaceprol, oxaprozin, pirprofen, pranoprofen, sulindac, suprofen, tiaprofenic acid, tinoridine acid, tolfenamic acid, tolmetin, xenbucin, ximoprofen, zaltoprofen, and zomepirac.

In some embodiments, the second agent or therapy is a cardioprotective therapy comprising a beta-blocker, a diuretic, an angiotensin-converting enzyme (ACE) inhibitor, a calcium channel blocker, a lipid-lowering therapy, a statin, a nitrate, an antiplatelet, an anticlotting agent, an anticoagulation agent, or combinations thereof.

In certain embodiments, the alternative anionic polymer is an anionic heteropolymer, or a pharmaceutically acceptable salt thereof. In certain embodiments, the anionic heteropolymer has two or more repeating units of acrylic acid, or a salt thereof; methacrylic acid, or a salt thereof, maleic acid, or a salt thereof; fumaric acid, or a salt thereof; ethylsulphonic acid, or a salt thereof; vinylsulphonic acid, or a salt thereof; styrenesulphonic acid, or a salt thereof; vinylphenylsulphuric acid, or a salt thereof; 2-methacryloyloxyethane sulphonic acid, or a salt thereof; 3-methacryloyloxy-2-hydroxypropanesulphonic acid, or a salt thereof; 3-methacryl amido-3-methylbutanoic acid, or a salt thereof; acrylamidomethylpropanesulfonic acid, or a salt thereof, vinylphosphoric acid, or a salt thereof; 4-vinylbenzoic acid, or a salt thereof; 3-vinyl oxypropane-1-sulphonic acid, or a salt thereof, or N-vinylsuccinimidic acid, or a salt thereof. In certain embodiments, anionic polymer is selected from the group consisting of: polystyrene sulfonic acid, or the sodium salt thereof, poly (styrene-co-maleic acid), or the sodium salt thereof; poly (2-acrylamido-2-methyl-1-propanesulfonic acid), or the sodium salt thereof, poly (styrene-alt-maleic acid), or the sodium salt thereof; and poly(4-styrenesulfonic acid-co-maleic acid), or the sodium salt thereof.

In certain embodiments, poly [4-styrenesulfonic acid-co-maleic acid] (PSCMA) or alternative anionic polymer anionic polymer comprises about 100 to about 10,000 constitutional repeating units or about 100 to about 20,000 constitutional repeating units.

In certain embodiments, the alternative anionic polymer is not dextran sulfate, dextran sulfate sodium, pentosan polysulfate, or pentosan polysulfate sodium.

In certain embodiments, poly [4-styrenesulfonic acid-co-maleic acid] (PSCMA) or alternative anionic polymer, or a pharmaceutically acceptable salt thereof is a pharmaceutically acceptable compositions in the form of, e.g., an aqueous solution, an oily preparation, a fatty emulsion, an emulsion, a lyophilized powder for reconstitution. The composition may be administered in the form of oral preparations (for example solid preparations such as tablets, caplets, capsules, granules, or powders; liquid preparations such as syrup, emulsions, dispersions, or suspensions).

In certain embodiments, poly [4-styrenesulfonic acid-co-maleic acid] (PSCMA) or alternative anionic polymer, or a pharmaceutically acceptable salt thereof may also include the use of one or more pharmaceutically acceptable additives, amino acids, polypeptides, polymers, solvents, buffers, excipients, preservatives, isotonic agents, diluents, stabilizers, and bulking agents.

Examples of suitable preservatives are benzoic acid esters of para-hydroxybenzoic acid, propylene glycol, phenols, phenylethyl alcohol or benzyl alcohol. Examples of suitable buffers are sodium phosphate salts, citric acid, tartaric acid, and the like. Examples of suitable stabilizers are, antioxidants such as alpha-tocopherol acetate, sodium metabisulphite, ascorbic acid, acetylcysteine, 8-hydroxyquinoline. Examples of suitable viscosity enhancing agents, suspending, or dispersing agents are substituted cellulose ethers, substituted cellulose esters, polyvinyl alcohol, polyvinylpyrrolidone, polyethylene glycols, carbomer, polyoxypropylene glycols, sorbitan monooleate, polyoxyethylene hydrogenated castor oil. Examples of suitable pH controllers include hydrochloric acid, sodium hydroxide and the like. Examples of suitable isotonic agents are glucose, D-sorbitol or D-mannitol, sodium chloride.

When administered parenterally, the compositions will normally be in a unit dosage, sterile, pyrogen free injectable form (solution, suspension, or emulsion, which may have been reconstituted prior to use), which is generally isotonic with the blood of the recipient with a pharmaceutically acceptable carrier. Examples of such sterile injectable forms are sterile injectable aqueous or oleaginous suspensions. These suspensions may be formulated according to techniques known in the art using suitable vehicles, dispersing, or wetting agents and suspending agents. The sterile injectable forms may also be sterile injectable solutions or suspensions in non-toxic parenterally acceptable diluents or solvents. Among the pharmaceutically acceptable vehicles and solvents that may be employed are water, ethanol, glycerol, saline, Ringer's solution, dextrose solution, isotonic sodium chloride solution, and Hanks' solution. In addition, sterile, fixed oils are conventionally employed as solvents or suspending mediums. For this purpose, any oil may be employed including synthetic mono-or di-glycerides, corn, cottonseed, peanut, and sesame oil. Fatty acids such as ethyl oleate, isopropyl myristate, and oleic acid and its glyceride derivatives, including olive oil and castor oil are useful in the preparation of injectables. These oil solutions or suspensions may also contain long-chain alcohol diluents or dispersants.

### Treating Traumatic Head Injury With Poly(4-styrenesulfonic acid-co-maleic acid) (PSCMA)

Chronic Traumatic Encephalopathy (CTE) is a neurodegenerative disease linked to repetitive head injuries (RHI), notably in athletes after contact sports and in soldiers after blast injury. Clinical Features can include impairments in mood, cognition, behavior, and neurologic function. CTE is a postmortem diagnosis, the hallmark of which is a tauopathy with neurofibrillary tangles found in the cortical sulci, medial temporal lobe, and brainstem.

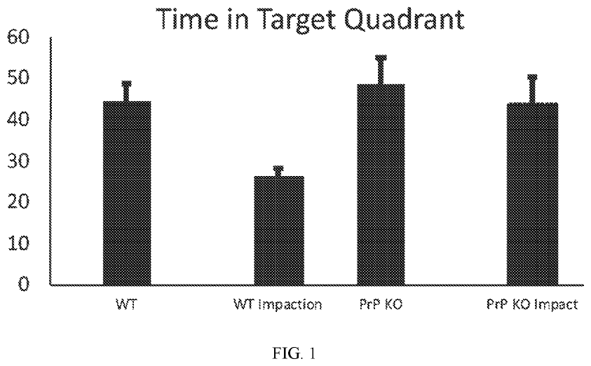

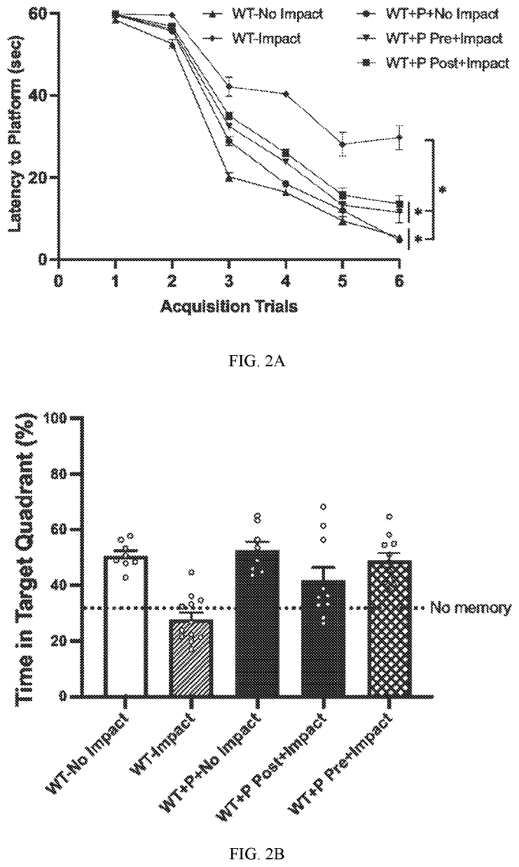

Tau isoforms are the same in CTE and AD. Beta Amyloid is present in 30% of postmortem acute TBI cases and 45% of CTE cases. PSCMA is an anionic polymer that inhibits AB oligomer binding to PrP, and experiments reported herein indicate that pretreatment with PSCMA Protects Against RHI (See FIGS. 1, 2A, and 2B).

### In Vivo Experiments in Mice

Three-month-old wildtype (WT) mice were assigned to five different cohorts. These included WT unimpacted, WT impacted, WT treated with PSCMA prophylactically before impaction, WT treated with PSCMA after the last impact, and WT unimpacted treated with PSCMA. Before impactions, the mice were swum in the Morris Water Maze to establish baseline performance. This included 6 groups of swims over three days. Each group is an average of 3 swims. Over the 6 groups of swims the mice were able to find the platform nicely. The animals were then subjected to 10 closed head injuries daily over ten days, alternating the side of the injury. Control (uninjured) animals were induced with anesthesia but where not impacted. The animals were then allowed to recover for 6 weeks. The animals were then swum in the Morris Water Maze to the opposite platform location (to preinjury). After the 6th trial group, the mice were given a 24-hour rest. The platform was then removed, and a probe trial was conducted. The mice were allowed to swim in the tank for 60 seconds and their location in the tank was tracked using a camera system.

For the prophylactic group, PSCMA was administered 1 week before the first swim trial and throughout the impactions, recovery, and reversal swims. For the post impaction treatment group PSCMA was administered after the last impaction, throughout recovery, and through the reversal swims. Method of administration was to mix the PSCMA with the mouse water.

In the reversal swims an approximately 70% improvement in memory with PSCMA administration was observed. This improvement was present in the both the mice treated prophylactically and the mice treated post-injury. Of note, the post-impaction PSCMA animals were impacted ten times over ten days and the PSCMA was administered after the last impaction. This implies that there is a treatment window for head injury that extends days post injury.

With the probe trial improvement was observed in both the prophylactic and post-injury treatment groups. The prophylactic group did slightly better than the post-injury treatment group. The prophylactic treatment group was statistically indistinguishable from uninjured controls.

What is claimed is:

1. A method of treating traumatic brain injury comprising administering an effective amount of poly [4-styrenesulfonic acid-co-maleic acid] (PSCMA) or a pharmaceutically acceptable salt thereof to a human patient in need thereof.

2. The method of claim 1, wherein the subject experienced a concussion and was unconscious for more than one second.

3. The method of claim 1, wherein the subject experienced a concussion and was unconscious for more than 10 seconds.

4. The method of claim 1, wherein the subject does not have a neurodegenerative disease.

5. The method of claim 1, wherein the subject does not have Alzheimer's disease.

6. The method of claim 1, wherein PSCMA is administered in combination with a second therapeutic agent.

7. The method of claim 6, wherein the second therapeutic agent is an anti-inflammatory agent.

8. The method of claim 6, wherein the second therapeutic agent is a glucocorticoid, dexamethasone, progesterone, or methylprednisolone.

9. The method of claim 6, wherein the second therapeutic agent is a non-steroidal anti-inflammatory drug, indomethacin, ibuprofen, carprofen, or celecoxib.

10. The method of claim 6, wherein the second therapeutic agent is a statin, simvastatin, atorvastatin, or lovastatin.

11. A method of treating chronic traumatic encephalopathy comprising administering an effective amount of poly [4-styrenesulfonic acid-co-maleic acid] (PSCMA) or a pharmaceutically acceptable salt thereof to a human patient in need thereof.

12. The method of claim 11, wherein the subject experienced a concussion and was unconscious for more than one second.

13. The method of claim 11, wherein the subject experienced head a concussion and was unconscious for more than 10 seconds.

14. The method of claim 11, wherein the subject does not have a neurodegenerative disease.

15. The method of claim 11, wherein the subject does not have Alzheimer's disease.

16. The method of claim 11, wherein PSCMA is administered in combination with a second therapeutic agent.

17. The method of claim 16, wherein the second therapeutic agent is an anti-inflammatory agent.

18. The method of claim 16, wherein the second therapeutic agent is a glucocorticoid, dexamethasone, progesterone, or methylprednisolone.

19. The method of claim 16, wherein the second therapeutic agent is a non-steroidal anti-inflammatory drug, indomethacin, ibuprofen, carprofen, or celecoxib.

20. The method of claim 16, wherein the second therapeutic agent is a statin, simvastatin, atorvastatin, or lovastatin.
